(19) United States
(12) Patent Application Publication
(10) Pub. No.: US 2013/0003019 A1
(43) Pub. Date: Jan. 3, 2013

(54) OPHTHALMOLOGIC APPARATUS AND OPHTHALMOLOGIC SYSTEM

(75) Inventor: Wataru Sakagawa, Kawasaki-shi (JP)

(73) Assignee: CANON KABUSHIKI KAISHA, Tokyo (JP)

(21) Appl. No.: 13/529,193

(22) Filed: Jun. 21, 2012

(30) Foreign Application Priority Data
Jul. 1, 2011 (JP) 2011-147126
May 29, 2012 (JP) 2012-121488

(51) Int. Cl. A61B 3/10

(52) U.S. Cl. 351/214; 351/246

(57) ABSTRACT

Provided is an ophthalmologic apparatus capable of measuring a cornea thickness with good accuracy over a wide range of cornea thickness distribution. The ophthalmologic apparatus is capable of measuring the cornea thickness of an eye to be inspected based on a width of an image of scattered light on a light receiving element for receiving light scattered in a cornea of the eye to be inspected, of slit light projected to the cornea, and an intensity of light received by the light receiving element.

### BACKGROUND OF THE INVENTION

1. Field of the Invention

The present invention relates to an ophthalmologic apparatus and an ophthalmologic system which have a function of measuring a cornea thickness.

2. Description of the Related Art

As a method for measuring a cornea thickness in a non-contact manner, there is known a measuring method using slit light. According to this measuring method, the slit light is projected to a cornea vertex of the eye to be inspected so that scattered light is generated inside the cornea. This scattered light is received by an image pickup element disposed on an optical axis having an angle different from that of an optical axis of the projected light so that a cornea tomographic image is obtained. One of edges of the cornea tomographic image corresponds to a cornea front surface, and the other corresponds to a cornea back surface. The width between the edges is different depending on the cornea thickness. Therefore, the edges of the cornea tomographic image are detected from output information of the image pickup element, and the width between the edges is substituted into an appropriate calculation equation. Thus, the cornea thickness can be determined.

In order to detect the edges of the cornea tomographic image, various image analysis methods are used, and there is known a technology of discriminating the cornea tomographic image from a disturbance light image (see Japanese Patent Application Laid-Open No. 2010-131333). The calculation equation of the cornea thickness is generated based on a measurement principle, and uses values such as a projection angle of the slit light, a light receiving angle, a magnification of a light receiving optical system, a refractive index and curvature of the cornea, and the like. Note that, in those documents, a width of the projected slit light is regarded to be small enough to be ignored (see Japanese Patent No. 4,349,937).

However, various error factors and conditions are added in reality, and hence there is considered a case where the calculation equation does not match the actual cornea thickness. For instance, the calculation equation is based on the precondition that the projected slit light has no width, but in reality, the slit light having a certain width is used. In particular, when the cornea is thin, this difference has a large influence. This is because a ratio of the width of the projected slit light to the width of the cornea tomographic image becomes larger as the cornea tomographic image obtained from the light receiving element becomes thinner. In recent years, in particular, there have been increasing subjects having a thin cornea because corneal refractive surgery has become widespread. Therefore, it is required to measure a thin cornea with high accuracy.

Therefore, the projected slit light having as small width as possible is used. However, when the width of the projected slit light is reduced, scatter in the cornea is also reduced. Therefore, luminance necessary for measuring the cornea tomographic image cannot be secured. It can be considered to solve the problem by using a high luminance light source or a high sensitivity image pickup element, but this solution is not desirable because this solution increases a cost of the apparatus.

### SUMMARY OF THE INVENTION

It is an object of the present invention to realize cornea thickness measurement with high accuracy over a wide range of cornea thickness distribution.

In order to achieve the above-mentioned object, an ophthalmologic apparatus according to an exemplary embodiment of the present invention includes: a slit light projecting optical system configured to project slit light to a cornea of an eye to be inspected; a slit light receiving optical system including a light receiving element for receiving scattered light of the slit light scattered in the cornea; and a cornea thickness measuring unit configured to measure a cornea thickness of the eye to be inspected based on a width of an image of the scattered light on the light receiving element and an intensity of light received by the light receiving element.

According to the exemplary embodiment of the present invention, it is possible to realize the cornea thickness measurement with high accuracy over a wide range of cornea thickness distribution.

Further features of the present invention will become apparent from the following description of exemplary embodiments with reference to the attached drawings.

### BRIEF DESCRIPTION OF THE DRAWINGS

FIG. 1A is a block diagram of an ophthalmologic apparatus of embodiments of the present invention.

FIG. 1B is a schematic diagram of a slit plate of the ophthalmologic apparatus of the embodiments of the present invention.

FIG. 2 is an internal block diagram of a system control portion 70.

FIG. 3A illustrates a cornea tomographic image taken by the ophthalmologic apparatus of the embodiments of the present invention.

FIG. 3B illustrates a measurement model example of a cornea thickness obtained from the cornea tomographic image illustrated in FIG. 3A.

FIG. 4A illustrates a cornea thickness measurement model example of the ophthalmologic apparatus of the embodiments of the present invention.

FIG. 4B illustrates a cornea thickness measurement model example of the ophthalmologic apparatus of the embodiments of the present invention.

FIG. 5 is a diagram illustrating an inspection flow.

FIG. 6 is a cornea thickness measurement flowchart of an ophthalmologic apparatus of a first embodiment of the present invention.

FIG. 7 is a cornea thickness measurement flowchart of an ophthalmologic apparatus of a second embodiment of the present invention.

### DESCRIPTION OF THE EMBODIMENTS

Hereinafter, embodiments of the present invention are described with reference to the attached drawings.

### First Embodiment

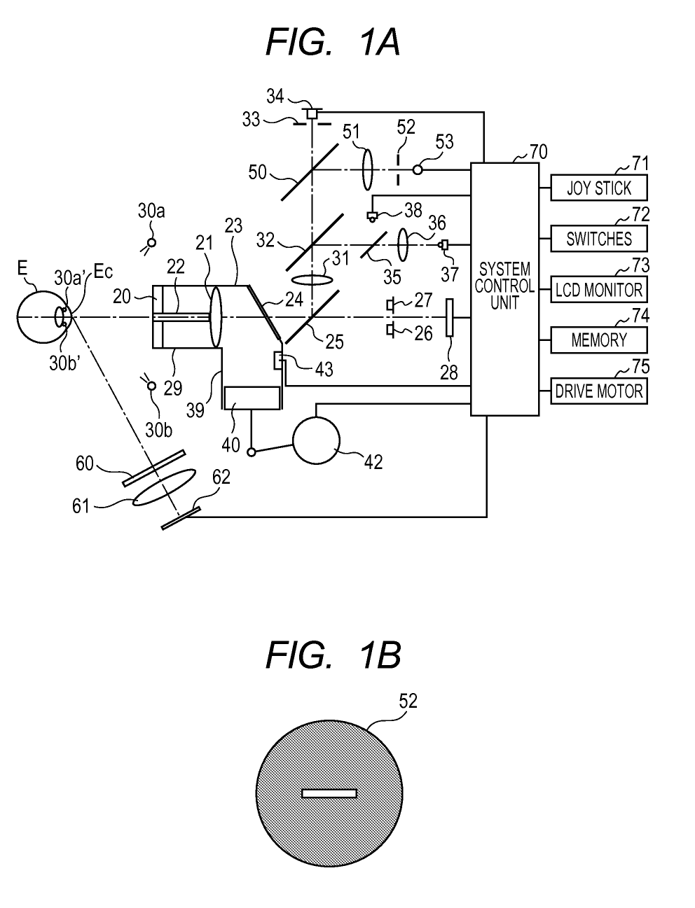

An ophthalmologic apparatus according to a first embodiment of the present invention is described with reference to FIG. 1A. Note that, the ophthalmologic apparatus according to this embodiment has a cornea thickness measurement function and an ocular pressure measurement function, but the ophthalmologic apparatus according to the present invention only needs to have the cornea thickness measurement function.

As opposed to a cornea Ec of an eye to be inspected E, there is disposed a nozzle 22 on the center axis of a parallel flat glass 20 and an objective lens 21. On the rear of the nozzle 22, an air chamber 23, an observation window 24, a dichroic mirror 25, a prism aperture stop 26, an imaging lens 27, and an image pickup element 28 are arranged in the stated order. Those members constitute a light receiving optical path of an observation optical system and an alignment detecting optical path for the eye to be inspected E.

The parallel flat glass 20 and the objective lens 21 are supported by an objective barrel 29, and outside the objective barrel 29, there are disposed external eye illumination sources 30a and 30b for illuminating the eye to be inspected E. Note that, for convenience of description, the external eye illumination sources 30a and 30b are disposed on the upper and lower sides in FIG. 1A, but in reality, the external eye illumination sources 30a and 30b are disposed perpendicularly to FIG. 1A so as to be opposed with respect to the optical axis.

In the reflection direction of the dichroic mirror 25, there are disposed a relay lens 31, a half mirror 32, a dichroic mirror 50 having characteristics of transmitting light having a near infrared wavelength and reflecting light having a visible light wavelength, an aperture 33, and a light receiving element 34. Note that, a position of the aperture 33 is set to be substantially conjugate with a cornea reflection image of a measuring light source 37 to be described later when a predetermined deformation occurs, and hence the aperture 33 constitutes a deformation detection light receiving optical system together with the light receiving element 34, which is used when the cornea Ec is deformed in an optical axis direction. The relay lens 31 is designed so as to form a cornea reflection image having substantially the same size as that of the aperture 33 when a predetermined deformation occurs in the cornea Ec.

In the incident direction of the half mirror 32, there are disposed a half mirror 35, a projection lens 36, and the light source 37 for ocular pressure measurement constituted of a near infrared LED that emits light having a wavelength of 880 nm. In the incident direction of the half mirror 35, there is disposed a fixation light source 38 constituted of an LED to be stared by a subject. The light source 37 for ocular pressure measurement also works as a light source for alignment of the eye to be inspected E. It is possible to dispose an independent light source for alignment, but it is preferred that the light source be shared for cost reduction.

In the incident direction of the dichroic mirror 50, there are disposed a projection lens 51, a slit plate 52, and a light source 53 that emits light having a wavelength of 465 nm for cornea thickness measurement.

The light source 53 illuminates the slit plate 52, and the image of the slit plate 52 is formed on the cornea Ec by the projection lens 51 and the relay lens 31 via the nozzle 22. The slit plate 52 has a rectangular aperture stop as illustrated in FIG. 1B, and is disposed so that the longitudinal direction thereof is perpendicular to the drawing sheet of FIG. 1A. Those members constitute a slit light projecting optical system for projecting slit light to the cornea of the eye to be inspected in the present invention.

In a diagonally lower direction of the eye to be inspected E, there are disposed a filter 60 that transmits light having a wavelength of 465 nm, an imaging lens 61, and an image pickup element 62, which constitute a light receiving optical system for cornea thickness measurement. The optical axis of the observation optical system and the optical axis of the light receiving optical system for cornea thickness measurement cross each other at the corneal vertex of the cornea Ec of the eye to be inspected. Therefore, the filter 60 transmits the cornea scattered light from the light source 53. The image pickup element 62 corresponds to a light receiving element in a slit light receiving optical system to be described later. In addition, the slit plate 52, the cornea Ec, and the image pickup element 62 have a substantially conjugate relationship. In other words, the configuration for receiving the cornea scattered light constitutes the slit light receiving optical system for receiving the scattered light in the cornea in the present invention.

In the air chamber 23, a piston 40 fits in a cylinder 39 constituting a part of the air chamber 23, and the piston 40 is driven by a solenoid 42. Note that, a pressure sensor 43 for monitoring internal pressure is disposed inside the air chamber 23.

The optical configuration of the apparatus of this embodiment is as described above.

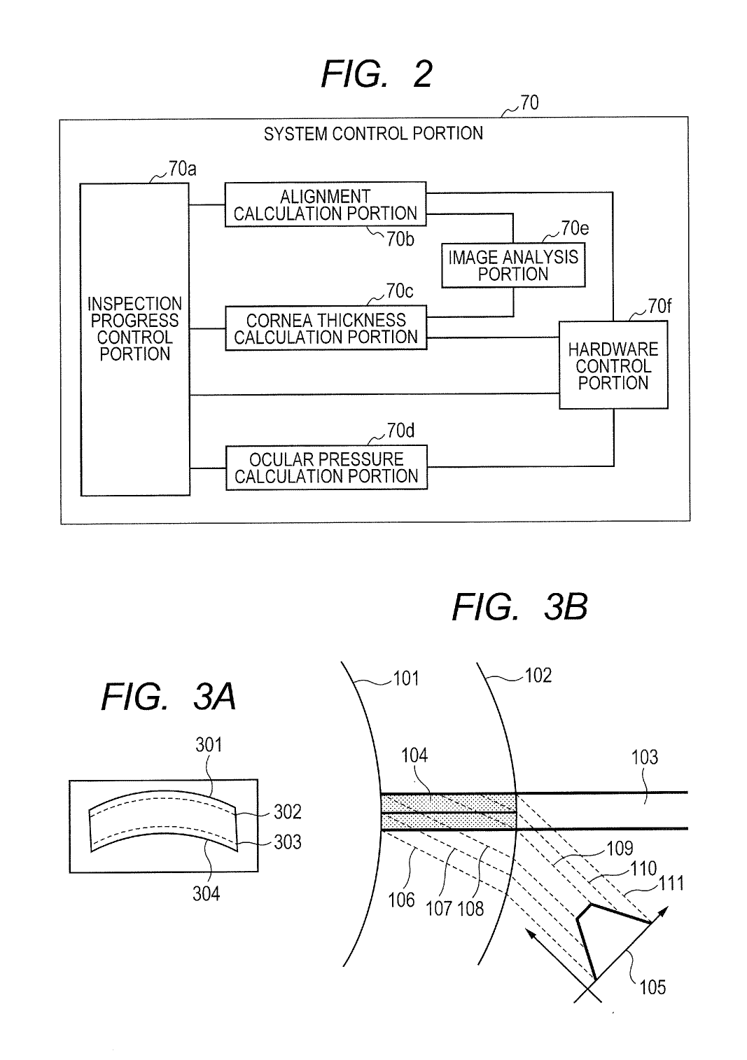

FIG. 2 is an internal block diagram of a system control portion 70. An inspection progress control portion 70a controls an alignment calculation portion 70b, a cornea thickness calculation portion 70c, an ocular pressure calculation portion 70d, and a hardware control portion 70f. Further, the alignment calculation portion 70b and the cornea thickness calculation portion 70c control an image analysis portion 70e and the hardware control portion 70f. The image analysis portion 70e analyzes images acquired by the image pickup element 28 and the image pickup element 62. The hardware control portion 70f controls the hardware portions illustrated in FIG. 1A.

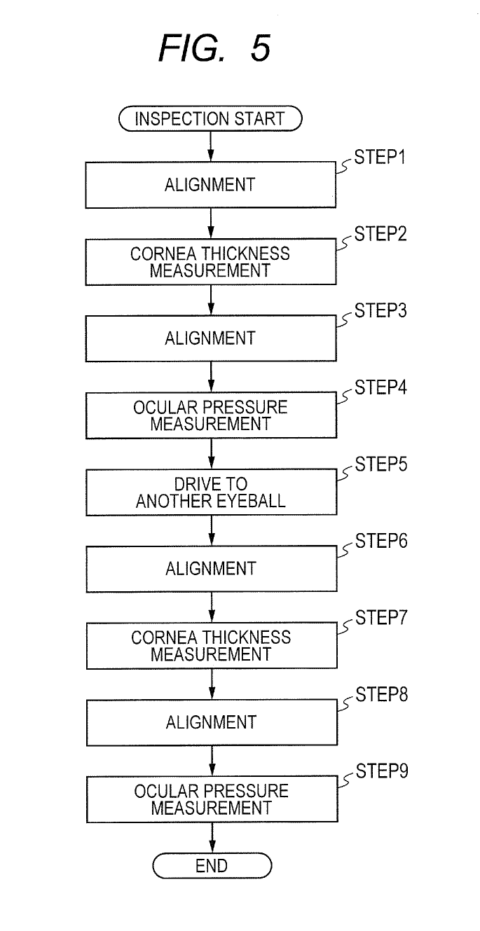

FIG. 5 is a diagram illustrating an inspection flow. First, the alignment calculation portion 70b performs alignment in STEP 1, and the cornea thickness calculation portion 70c performs the cornea thickness measurement in STEP 2. After that, the alignment calculation portion 70b performs the alignment again in STEP 3, and the ocular pressure calculation portion 70d performs the ocular pressure measurement in STEP 4. The system control portion 70 controls driving to another eyeball in STEP 5, then measures the another eyeball similarly, and ends the inspection.

Next, a system configuration and operation of the apparatus of this embodiment are described.

The alignment calculation portion 70b first causes the eye to be inspected E to stare the fixation light source 38. In this state, an examiner presses a measurement start switch 72. When the measurement start switch 72 is pressed, the light source 37 for ocular pressure measurement is activated. The light beam from the light source 37 for ocular pressure measurement is collimated by the projection lens 36, is reflected by the half mirror 32, and an image thereof is once formed in the nozzle 22 by the relay lens 31. Then, the light beam irradiates the cornea Ec of the eye to be inspected E. A corneal bright spot of the image formed by the cornea Ec is split by the prism aperture stop 26 and is acquired by the image pickup element 28. An output from the image pickup element 28 is displayed on a monitor 73 by the hardware control portion 70f. The hardware control portion 70f drives a drive motor 75 based on a positional relationship of the split corneal bright spots, drives a main body portion (not shown), and performs automatic alignment in X, Y, and Z axis directions.

Note that, the alignment may be performed manually. In this case, the examiner operates a joy stick 71 while observing the corneal bright spot displayed on the monitor 73, and the hardware control portion 70f drives the drive motor 75 according to the input of the joy stick 71. When the positional relationship of the corneal bright spots becomes a predetermined state, the alignment is completed.

The cornea thickness calculation portion 70c performs the cornea thickness measurement. The hardware control portion 70f drives the light source 53 with pulse so that the slit light illuminates the cornea Ec. An image of the light scattered in the cornea Ec is taken by the image pickup element 62. The cornea tomographic image taken by the image pickup element 62 is illustrated simply in FIG. 3A. The output information from the image pickup element 62 is stored in a memory 74 by the hardware control portion 70f. The cornea thickness calculation portion 70c calculates the cornea thickness based on information on the cornea tomographic image stored in the memory 74, and a result of the measurement is displayed on the monitor 73.

The ocular pressure calculation portion 70b performs the ocular pressure measurement. The hardware control portion 70f drives the solenoid 42 so that the air in the air chamber 23 is compressed by the piston 40 pushed up by the solenoid 42. Thus, a pulse-like air flow issues from the nozzle 22 to the cornea Ec of the eye to be inspected E. A pressure signal detected by the pressure sensor 43 in the air chamber 23 and the light receiving signal from the light receiving element 34 are stored in the memory 74 by the hardware control portion 70f. The ocular pressure calculation portion 70b calculates an ocular pressure value from a peak value of the light receiving signal and a pressure signal at the time based on information stored in the memory 74. The calculated ocular pressure value is displayed on the monitor 73 by the hardware control portion 70f.

The apparatus configuration of this embodiment is as described above. The configuration for calculating the ocular pressure value described above constitutes an ocular pressure measuring unit in the present invention. The ocular pressure measuring unit is corrected based on an output concerning the cornea thickness obtained by a cornea thickness measuring unit to be described later. The corrected ocular pressure value y (mmHg) is calculated based on the correction equation of y=x+a(b−t), where x (mmHg) represents the measured ocular pressure value and t (μm) represents the measured cornea thickness, and further, a and b are constants, which can be changed arbitrarily by the examiner. When the examiner does not change the constants, default values stored in the apparatus are used. As default values, for example, a=0.045 and b=554 are stored. It is preferred that the ocular pressure measuring unit have a configuration for performing the above-mentioned correction operation as a correcting unit, and the correcting unit is included in the ocular pressure calculation portion 70d. The correcting unit may perform the correction by a method different from the above-mentioned correction equation.

Details of the cornea thickness measurement of the present invention are described below.

Supposing that a scattering amount in the cornea is uniform, the cornea tomographic image illustrated in FIG. 3A is obtained according to a principle illustrated in FIG. 3B. FIG. 3B illustrates a manner in which a slit light 103 is scattered between a cornea front surface 102 and a cornea back surface 101. A light intensity distribution 105 indicates a distribution of light intensity depending on a light receiving position, namely, a profile of the cornea tomographic image. A horizontal axis represents the light receiving position, and a vertical axis represents the light intensity.

A rectangular portion 104 in which the slit light crosses the cornea is a scattering portion, and optical paths from the scattering portion to a light receiving system are indicated by dot lines. An optical path 106 corresponds to a solid line 304, an optical path 108 corresponds to a dot line 303, an optical path 109 corresponds to a dot line 302, and an optical path 111 corresponds to a solid line 301. The optical paths have light intensities proportional to lengths of the scattering portions to pass through. Specifically, the optical path 106 and the optical path 111 that do not pass through the scattering portion have no light intensity. The optical path 108 to the optical path 109 having the longest length of the scattering portion to pass through have a maximum light intensity. In addition, an optical path 110 from the center of the cornea front surface and an optical path 107 from the center of the cornea back surface have a half light intensity of the maximum light intensity. Therefore, a distance between the center of the cornea front surface and the center of the cornea back surface corresponds to a distance between the optical path 110 and the optical path 107. In other words, by a binarization process with respect to a half of the maximum light intensity as a threshold value, the distance between the optical path 110 and the optical path 107 can be determined.

By performing calculation on this distance using a slit light projection angle, a light receiving angle, a light receiving magnification, and a refractive index and curvature of the cornea, the cornea thickness can be calculated. Among the values to be used for this calculation, values unique to the apparatus may be known values of design or may be values reflecting individual variation among the apparatus. In addition, values concerning the cornea may be known values as properties of the cornea, an input unit for inputting those values may be added, or measuring units for measuring the respective values may be disposed so that measured values are reflected. In addition, it is possible to adopt a measuring unit for performing calculation by using various values for reflecting a more actual situation other than the above-mentioned values.

The above-mentioned configuration, including a converting unit for performing binarization process with respect to a half of the maximum light intensity as the threshold value and a calculating unit for calculating the cornea thickness based on the binary boundaries obtained by the converting unit, corresponds to a first cornea thickness measuring unit of this embodiment. Note that, the first cornea thickness measuring unit including the converting unit and the calculating unit is included in the cornea thickness calculation portion 70c. Further, in other words, when the width of the light image received by the light receiving element is a predetermined value or more, the first cornea thickness measuring unit measures the cornea thickness based on the width of the image.

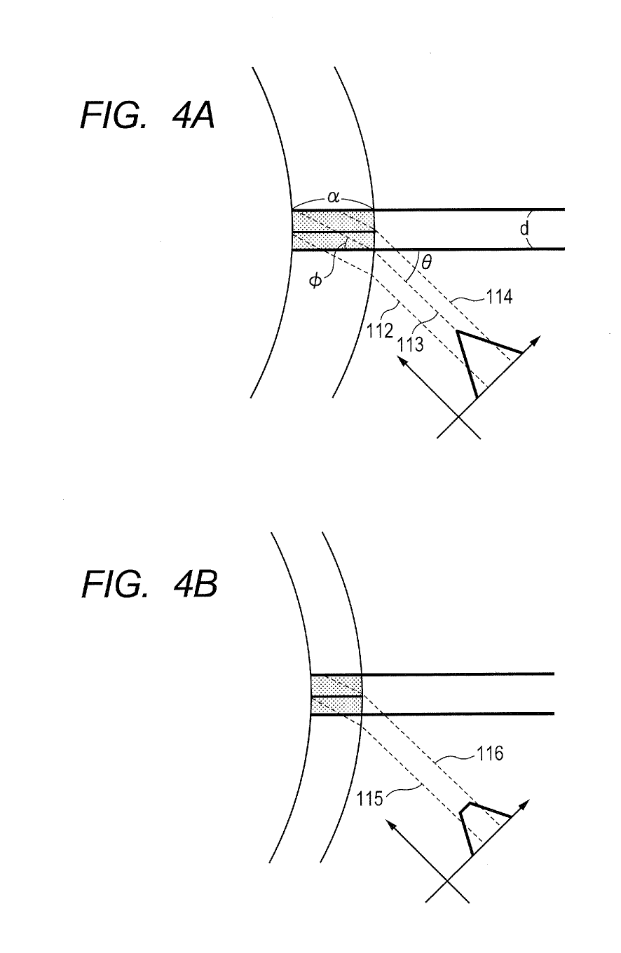

When the thickness of the cornea is less than that illustrated in FIG. 3B, the width between the optical path 108 and the optical path 109 becomes small. When the width between the optical path 108 and the optical path 109 becomes zero, the both optical paths are identical to each other as indicated by an optical path 113 of FIG. 4A. In other words, the optical path 113 travels on a diagonal line of the scattering portion 104, and hence there is no other optical path having the same light intensity as that of the optical path 113 so that the light intensity distribution has a triangular shape. In this case, the maximum light intensity is the same as that illustrated in FIG. 3B, and hence the cornea thickness can be calculated by measuring a width between an optical path 114 from the center of the cornea front surface and an optical path 112 from the center of the cornea back surface. In other words, as long as the cornea thickness has a value in the range from the light intensity distribution illustrated in FIG. 3B to the light intensity distribution illustrated in FIG. 4A, the cornea thickness can be calculated by the above-mentioned principle.

However, when the cornea becomes thinner than that illustrated in FIG. 4A, the shape of the light intensity distribution becomes a trapezoid again as illustrated in FIG. 4B, but the maximum light intensity in the light intensity distribution becomes lower than that illustrated in FIG. 3B or 4A. In this case, light intensities of an optical path 116 from the cornea front surface and an optical path 115 from the cornea back surface are larger than a half of the maximum light intensity. Therefore, when the binarization process is performed with respect to a half of the maximum light intensity as the threshold value, the cornea thickness is calculated to be more than the real value.

On the other hand, when the cornea is thinner than that illustrated in FIG. 4A, the maximum light intensity in the scattering portion and the cornea thickness have a positive correlation. When the scattering portion 104 is approximated by a rectangle, the maximum light intensity in the scattering portion and the cornea thickness are proportional to each other. Therefore, when the cornea is thin as illustrated in FIG. 4B, the cornea thickness can be calculated based on the maximum light intensity in the scattering portion instead of calculating the cornea thickness based on the width between the optical paths. A cornea thickness y (μm) can be expressed as follows by using a maximum light intensity x in the cornea tomographic image acquired by the image pickup element 62:

$$y = \frac{α}{P} x \qquad Equation 1$$

where α (μm) represents a reference cornea thickness that is the cornea thickness illustrated in FIG. 4A, and P represents a reference light intensity that is an average maximum light intensity in the cornea tomographic image in this case. The reference light intensity P has a value determined by the light intensity of the light source 53, a diameter of the lens 61, sensitivity of the image pickup element 62, and the like, and is stored in the apparatus. The reference cornea thickness a is different depending on an apparatus configuration. In FIG. 4A, supposing that the scattering portion 104 has a rectangular shape, the following equation holds from Snell's law:

sin θ=n sin φ Equation 2

where θ represents an angle between the optical axis of the slit light projecting optical system and the optical axis of the slit light receiving optical system, y represents an angle between the optical axis of the slit light projecting optical system and the scattered light 113 at the cornea back surface, n represents a refractive index of the cornea with respect to the air at a wavelength of 465 nm.

In addition, the following equation also holds from properties of trigonometric ratios:

$$α = \frac{d}{tan ϕ} \qquad Equation 3$$

where d (μm) represents a width of the slit light 103.

In this case, supposing that the refractive index n is 1.3828, for example, when θ is 55° and d is 100 (μm), α is 136 (μm). Here, the cornea thickness is usually in a range of 400 to 650 nm, even when the cornea thickness becomes thin as a result of the corneal refractive surgery. Therefore, when the reference cornea thickness α is 136 μm, the usual cornea thickness is more than α. However, in order to keep the slit light receiving optical system away from the face of the subject, it is preferred to set θ to be as small as possible. In addition, in order to set the light intensity of the slit light to be high, it is preferred to set a width d of the slit light to be as large as possible. From those viewpoints, for example, it is preferred to set θ to 20° and d to 150 (μm) in design. In this case, a becomes 588 (μm). This value is within the above-mentioned range from 400 to 650 nm. Therefore, it is necessary to assume the case illustrated in FIG. 4B, for example.

In addition, in order to perform more accurate calculation, it is possible to use a calculation equation other than the above-mentioned equation because the scattering portion 104 is not rectangular. In addition, it is possible to use different calculation equations depending on optical configurations of the apparatus.

The above-mentioned configuration including a second calculating unit for calculating the cornea thickness based on the maximum light intensity in the scattering portion is a second cornea thickness measuring unit of this embodiment. Note that, this second cornea thickness measuring unit including the second calculating unit is included in the cornea thickness calculation portion 70c. Note that, in the present invention, the first cornea thickness measuring unit measures the cornea thickness based on the width of the image of the scattered light in the cornea, which is formed on the light receiving element, while the second cornea thickness measuring unit measures the cornea thickness based on the light intensity of the light image formed on the light receiving element, and hence those measuring units constitute the cornea thickness measuring unit of the present invention. In addition, when the width of the light image received by the light receiving element is less than a predetermined value, the second cornea thickness measuring unit measures the cornea thickness based on the light intensity of the image. Here, whether or not to use the second cornea thickness measuring unit is determined by a width determination unit for determining whether or not the width of the light image is less than a predetermined value, and a used output determination unit for determining the measuring unit to be used based on a result of determination of the width determination unit. Therefore, it is possible to define that the cornea thickness measuring unit measures the cornea thickness of the eye to be inspected based on a result of determination of the used output determination unit. Each of the width determination unit and the used output determination unit is constituted of a module region of the cornea thickness calculation portion 70c, for performing the corresponding operation.

It is known in general that many human corneas scatter more light on the surface than in other portions, and the distribution thereof is not uniform and hence does not have a trapezoidal profile as in the above-mentioned assumption. However, in this case too, by storing the maximum light intensities at the optical path 108 and the optical path 109 so as to use two threshold values, it is possible to calculate the cornea thickness in the same manner as in the above-mentioned first cornea thickness measuring unit and the second cornea thickness measuring unit. In addition, it is possible to use another method as long as the method is suitable for the scattering intensity distribution of the human cornea. For instance, in the above-mentioned first cornea thickness measuring unit, when determining the distance between the optical path 110 and the optical path 107, it is possible to use an edge detection process or the like instead of the binarization process.

This configuration for measuring the cornea thickness by another method can be also defined as the cornea thickness measuring unit. It is preferred to add the region for performing the operation to the cornea thickness calculation portion 70c so that the ophthalmologic apparatus of the present invention has a plurality of cornea thickness measuring units including the first cornea thickness measuring unit or the second cornea thickness measuring unit described above. Further, in this case, according to a result of measurement by the first cornea thickness measuring unit, another cornea thickness measuring unit such as the second cornea thickness measuring unit is used according to the measurement flow to be described later. Thus, an appropriate result of measurement of the cornea thickness can be obtained.

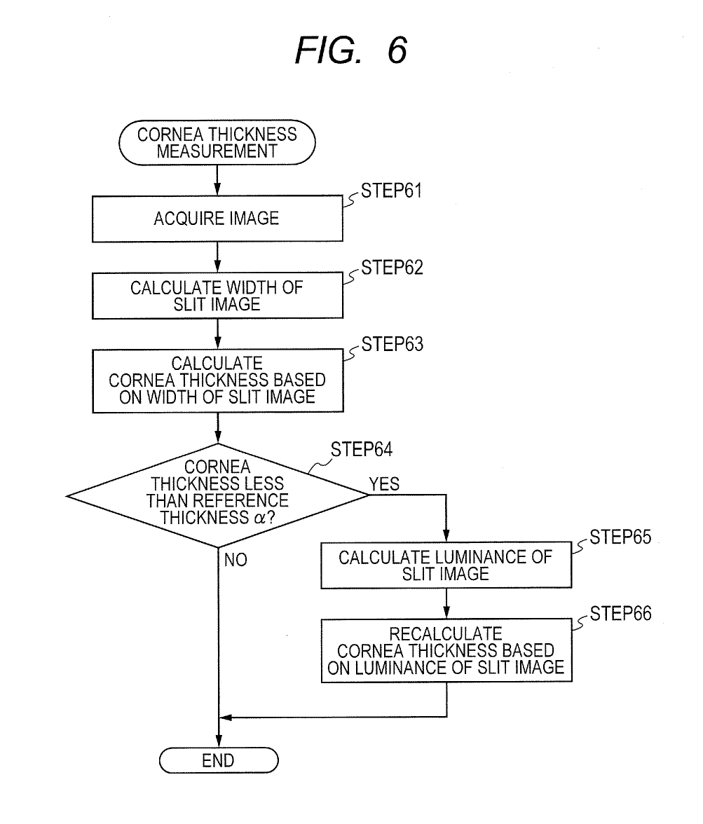

The first cornea thickness measuring unit and the second cornea thickness measuring unit described above can be used in the flow illustrated in FIG. 6. First, the image analysis portion 70e acquires the cornea tomographic image of the eye to be inspected in STEP 61, and calculates the width of the cornea tomographic image in STEP 62. Based on the width of the cornea tomographic image calculated in STEP 62, the cornea thickness calculation portion 70c calculates the cornea thickness in STEP 63. The first cornea thickness measuring unit in this embodiment is as described above. After that, in STEP 64, the cornea thickness calculation portion 70c determines whether or not the result of measurement is less than the reference cornea thickness a, namely whether or not the result of measurement is less than a predetermined value, by the determination unit described above. When the result of measurement is the reference cornea thickness a or more, the cornea thickness measurement ends. When the result of measurement is less than the reference cornea thickness a, the image analysis portion 70e calculates luminance of the cornea tomographic image in STEP 65. In STEP 66, the cornea thickness calculation portion 70c recalculates the cornea thickness based on the luminance of the cornea tomographic image calculated in STEP 65, and the cornea thickness measurement ends. The second cornea thickness measuring unit in this embodiment is as described above. According to this flow, it is possible to always perform the cornea thickness measurement with good accuracy over a wide cornea thickness distribution. Note that, in the measurement flow described above, in this embodiment, the determination by the determination unit is performed based on the width between the optical path 108 and the optical path 109 with use of the case where the width becomes zero as a reference. However, it is possible to set the reference to the case where the width has a predetermined value or lower, for example, or it is further possible to set the reference in consideration of the luminance value or the like. In addition, in the measurement flow described above, the luminance is calculated after the width of the cornea tomographic image is calculated, but it is possible to adopt the opposite order. In other words, it is possible to adopt the measurement flow in which the luminance of the cornea tomographic image is first calculated, and when the luminance is the reference value (predetermined value) or more, the width of the cornea tomographic image is calculated.

According to the present invention, the output from the light receiving element to be used for measuring by the first cornea thickness measuring unit is checked by the determination unit first, and when the output is an appropriate output, there is no need to use another measuring unit. However, when it is determined that the another cornea thickness measuring unit is more appropriate based on the determination result of the determination unit, an appropriate result of measurement can be obtained by measuring by the another cornea thickness measuring unit. Therefore, it is possible to always perform the cornea thickness measurement with good accuracy over the wide range of the cornea thickness distribution.

### Second Embodiment

When the cornea thickness is calculated from the luminance of the cornea tomographic image, measurement accuracy can be improved by considering individual variation of scattering ratio of the cornea. A second embodiment of the present invention is an embodiment in which scattering ratio information of the cornea of the subject can be stored in addition to the configuration of the first embodiment.

The examiner inputs an identity number of the subject by using the joy stick 71 and various switches 72 in the first stage of the inspection. The identity number may be an integer or a character string. In addition, it is possible to use an input unit such as an external device that is not included in FIG. 1A.

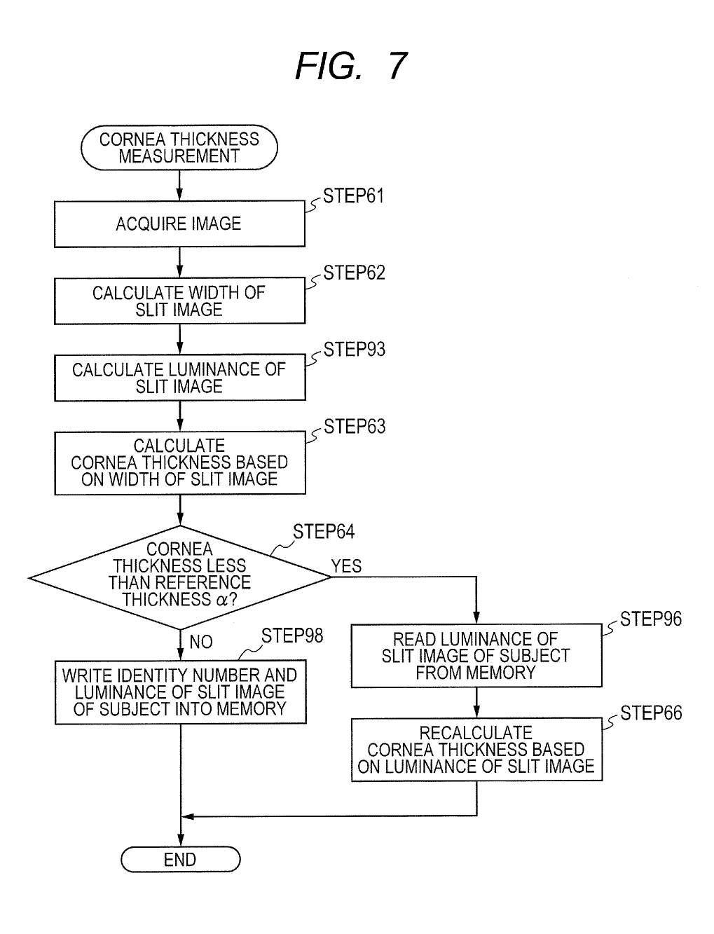

FIG. 7 is a flow of measurement of the cornea thickness in the second embodiment. Note that, the same step as in the measurement flow described above in the first embodiment is represented by the same step number, and overlapping description is omitted. The image analysis portion 70e calculates the luminance of the cornea tomographic image (maximum light intensity) in STEP 93. When the cornea thickness is the reference cornea thickness a or more (the case of FIG. 3B or 4A), the identity number of the subject and the luminance of the cornea tomographic image are stored together in the memory 74 in STEP 98. When the cornea thickness is less than the reference cornea thickness a, the cornea thickness calculation portion 70c reads the luminance of the cornea tomographic image of the subject from the memory 74 in STEP 96. In STEP 66, the cornea thickness calculation portion 70c performs the same calculation as that in STEP 66 of FIG. 6. This embodiment has a feature that in STEP 96, when the luminance of the cornea tomographic image of the subject is stored, the stored luminance of the cornea tomographic image is used as the reference light intensity P. Note that, when the luminance of the cornea tomographic image is not stored, a fixed value is used as the reference light intensity P similarly to the first embodiment. Presence or absence of the scattering ratio information of the cornea constituting the information about luminance is determined by the module region having the function as an information determination unit in the cornea thickness calculation portion 70c.

Note that, it is preferred that the scattering ratio information of the cornea of the subject be the luminance of the cornea tomographic image before the corneal refractive surgery. In addition, it is preferred that the scattering ratio information of the cornea of the subject include the light intensity of the slit light that is actually output in addition to the luminance of the cornea tomographic image. In this case, when the cornea measuring apparatus is different between the past and the present, more accurate reference light intensity can be used by using a ratio between the stored light intensity and the current light intensity of the slit light. However, when the cornea measuring apparatus is the same between the past and the present, it is sufficient to use the luminance of the cornea tomographic image as the scattering ratio information of the cornea of the subject.

In other words, in this embodiment, the cornea thickness measuring unit measures the cornea thickness of the eye to be inspected based on the obtained width of the cornea tomographic image, the light intensity, and the scattering ratio information of the cornea of the subject. In addition, the information about the scattering ratio of the cornea in this case is determined based on the luminance value obtained in the case where the width of the cornea tomographic image obtained as described above is a predetermined value or more.

As described above, in the case where the measurement was performed in the past in a sufficiently thick cornea state, and the measurement is performed again in a thinner cornea state, more accurate measurement value can be calculated by using the scattering ratio of the cornea in the past measurement. Therefore, it is possible to perform accurate measurement of the subject having a cornea that has become thinner as a result of a corneal refractive surgery or the like.

### Another Embodiment

Further, the present invention is also implemented by executing the following processing. Specifically, in this processing, software (program) for implementing the functions of the above-mentioned embodiments is supplied to a system or an apparatus via a network or various kinds of storage medium, and a computer (or CPU, MPU, etc.) of the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2011-147126, filed Jul. 1, 2011, and No. 2012-121488, filed May 29, 2012 which are hereby incorporated by reference herein in their entirety.

What is claimed is:

1. An ophthalmologic apparatus, comprising:
a slit light projecting optical system configured to project slit light to a cornea of an eye to be inspected;
a slit light receiving optical system including a light receiving element for receiving scattered light of the slit light scattered in the cornea; and
a cornea thickness measuring unit configured to measure a cornea thickness of the eye to be inspected based on a width of an image of the scattered light on the light receiving element and an intensity of light received by the light receiving element.

2. An ophthalmologic apparatus according to claim 1, wherein the cornea thickness measuring unit measures the cornea thickness of the eye to be inspected based on the intensity of light received by the light receiving element when the width of the image of the scattered light on the light receiving element is less than a predetermined value.

3. An ophthalmologic apparatus according to claim 1, wherein the cornea thickness measuring unit measures the cornea thickness of the eye to be inspected based on the width of the image of the scattered light on the light receiving element when the width is a predetermined value or more.

4. An ophthalmologic apparatus according to claim 1, wherein the cornea thickness measuring unit measures the cornea thickness of the eye to be inspected based on the width of the image of the scattered light on the light receiving element, the intensity of light received by the light receiving element, and scattering ratio information of the cornea of the eye to be inspected.

5. An ophthalmologic apparatus according to claim 4, wherein the scattering ratio information of the cornea of the eye to be inspected comprises information based on the intensity of light received by the light receiving element when the width of the image of the scattered light on the light receiving element is a predetermined value or more.

6. An ophthalmologic apparatus according to claim 4, wherein the cornea thickness measuring unit measures the cornea thickness by using a reference light intensity calculated based on a light intensity of the image of the scattered light received by the light receiving element.

7. An ophthalmologic apparatus according to claim 6, further comprising an information determination unit configured to determine presence or absence of the scattering ratio information of the cornea of the eye to be inspected,
wherein the cornea thickness measuring unit measures the cornea thickness by using the scattering ratio information of the cornea as the reference light intensity when the information determination unit determines that the scattering ratio information of the cornea of the eye to be inspected is present.

8. An ophthalmologic apparatus according to claim 1, wherein the cornea thickness measuring unit includes:
a first cornea thickness measuring unit configured to measure the cornea thickness of the eye to be inspected based on the width of the image of the scattered light on the light receiving element; and
a second cornea thickness measuring unit configured to measure the cornea thickness of the eye to be inspected based on a light intensity of the image of the scattered light on the light receiving element.

9. An ophthalmologic apparatus according to claim 8, further comprising a used output determination unit configured to determine whether or not to use the second cornea thickness measuring unit based on an output from the light receiving element,
wherein the cornea thickness measuring unit measures the cornea thickness of the eye to be inspected based on a result of determination by the used output determination unit.

10. An ophthalmologic apparatus according to claim 1, further comprising a width determination unit configured to determine whether or not the width of the image of the scattered light on the light receiving element is less than a predetermined value,
wherein the cornea thickness measuring unit measures the cornea thickness of the eye to be inspected based on a result of determination by the width determination unit.

11. An ophthalmologic apparatus according to claim 1, further comprising:
an ocular pressure measuring unit configured to measure an ocular pressure of the eye to be inspected; and
a correcting unit configured to correct the ocular pressure based on an output of the cornea thickness measuring unit.

12. An ophthalmologic system, comprising:
a slit light projecting optical system configured to project slit light to a cornea of an eye to be inspected;
a slit light receiving optical system including a light receiving element for receiving scattered light of the slit light scattered in the cornea; and
a cornea thickness measuring unit configured to measure a cornea thickness of the eye to be inspected based on a width of an image of the scattered light on the light receiving element and an intensity of light received by the light receiving element.

13. A cornea thickness measuring method, comprising:
projecting slit light to a cornea of an eye to be inspected;
receiving, by a light receiving element, scattered light of the slit light scattered in the cornea; and
measuring a cornea thickness of the eye to be inspected based on a width of an image of the scattered light on the light receiving element and an intensity of light received by the light receiving element.

14. A storage medium having a program stored thereon for causing a computer to perform the steps of the cornea thickness measuring method according to claim 13.
